(19) United States
(12) Patent Application Publication
(10) Pub. No.: US 2023/0054258 A1
(43) Pub. Date: Feb. 23, 2023

(54) METHODS, SYSTEMS, AND COMPUTER PROGRAM PRODUCTS FOR DETERMINING THIRD PARTY LIABILITY FOR A CLAIM

(71) Applicant: Change Healthcare Holdings LLC, Nashville, TN (US)

(72) Inventors: Mike Peresie, Milton, GA (US); Ed Hafner, Boerne, TX (US); Cindy Klain, Mansfield, MA (US)

(21) Appl. No.: 17/408,895

(22) Filed: Aug. 23, 2021

(51) Int. Cl. G06Q 40/08; G16H 10/60; G16H 50/20

(52) U.S. Cl. CPC G06Q 40/08; G16H 10/60; G16H 50/20

(57) ABSTRACT

A method includes receiving a claim associated with a patient and destined for a first payor from a provider; assessing a likelihood that the first payor is not liable for an entirety of the claim; identifying a second payor responsive to the likelihood that the first payor is not liable for the entirety of the claim exceeding a liability threshold; and routing the claim to the first payor, to the second payor, or to the provider based on routing rules provided by the first payor.

### FIELD

The present inventive concepts relate generally to health care systems and services and, more particularly, to processing of claims generated by providers for payment by payors.

### BACKGROUND

Health care service providers have patients that pay for their care using a variety of different payors. For example, a medical facility or practice may serve patients that pay by way of different insurance companies including, but not limited to, private insurance plans, government insurance plans, such as Medicare, Medicaid, and state or federal public employee insurance plans, and/or hybrid insurance plans, such as those that are sold through the Affordable Care Act. When providers submit claims to the payors for payment, the claims will often be paid by the payor to which the claim is sent. In some circumstances, however, a patient may be covered by another payor. For example, a patient may also be covered under a spouse's insurance policy. Or a patient may be covered under a government insurance plan, such as Medicare or Medicaid, but the patient may also be covered under a supplemental plan or under a spouse's plan. If the patient has been treated for injuries caused by an auto accident, for example, the patient's treatment may also be covered by an auto insurance policy. The payor typically has an audit or fraud and abuse department that reviews paid claims to determine whether the claims have been paid properly or whether there are any irregularities associated with payment of these claims. These post payment audits or reviews may determine that another payor is liable for at least a portion of the claim. The payor may then report this liability to the provider and seek reimbursement for the portion of the claim for which the payor is not responsible. This post payment review and reimbursement process, however, is generally a manual process that may be costly and labor intensive.

### SUMMARY

According to some embodiments of the inventive concept, a method comprises: receiving a claim associated with a patient and destined for a first payor from a provider; assessing a likelihood that the first payor is not liable for the entirety of the claim; identifying a second payor responsive to the likelihood that the first payor is not liable for the entirety of the claim exceeding a liability threshold; and routing the claim to the first payor, to the second payor, or to the provider based on routing rules provided by the first payor.

In still other embodiments, assessing the likelihood that the first payor is not liable for the entirety of the claim comprises: assessing the likelihood that the first payor is not liable for the entirety of the claim based on a plurality of patient coverage factors. The plurality of patient coverage factors comprises: an age of the patient, a spousal relationship status of the patient, information from historic claims associated with the patient, employment information for the patient and/or a spouse of the patient, an income level of the patient, and/or stored payor eligibility information for the patient.

In still other embodiments, identifying the second payor comprises: identifying the second payor based on a plurality of payor identification factors. The plurality of payor identification factors comprises: an age of the patient, a spousal relationship status of the patient, information from historic claims associated with the patient, employment information for the patient and/or a spouse of the patient, an income level of the patient, and/or stored payor eligibility information for the patient.

In still other embodiments, identifying the second payor comprises: identifying a plurality of additional payors based on a plurality of payor identification factors responsive to the likelihood that the first payor is not liable for the entirety of the claim exceeding a liability threshold. The plurality of payor identification factors comprises: an age of the patient, a spousal relationship status of the patient, information from historic claims associated with the patient, employment information for the patient and/or a spouse of the patient, an income level of the patient, and/or stored payor eligibility information for the patient.

In still other embodiments, the method further comprises confirming with the plurality of additional payors that the additional payors are responsible for at least portions of the claim, respectively.

In still other embodiments, confirming with the plurality of additional payors that the additional payors are responsible for at least portions of the claim, respectively, comprises: assigning a confidence score value to each of the plurality of additional payors based on a likelihood that the respective one of the additional payors is responsible for a respective portion of the claim; and communicating patient insurance coverage eligibility requests to ones of the additional payors, respectively, having confidence score value magnitudes that exceed a confidence threshold. A magnitude of the confidence score value is greater with increased likelihood of being responsible for the respective portion of the claim.

In still other embodiments, the method further comprises: confirming with the second payor that the second payor is responsible for at least a portion of the claim.

In still other embodiments, confirming with the second payor that the second payor is responsible for at least the portion of the claim comprises: communicating a patient insurance coverage eligibility request to the second payor; and receiving an insurance coverage confirmation reply from the second payor for the patient when the patient has insurance coverage provided by the second payor.

In still other embodiments, routing the claim to the first payor, to the second payor, or to the provider based on the routing rules provided by the first payor comprises: routing the claim to the first payor with a flag that identifies the second payor as being responsible for at least the portion of the claim.

In still other embodiments, routing the claim to the first payor, to the second payor, or to the provider based on the routing rules provided by the first payor comprises: routing the claim to the provider notifying the provider that the first payor is not responsible for the entirety of the claim.

In still other embodiments, routing the claim to the first payor, to the second payor, or to the provider based on the routing rules provided by the first payor comprises: routing the claim to the second payor.

In still other embodiments, the method further comprises: notifying the first payor that the claim has been routed to the second payor.

In still other embodiments, routing the claim to the first payor, to the second payor, or to the provider based on the routing rules provided by the first payor comprises: assigning a confidence score value to the second payor based on a likelihood that the second payor is responsible for the at least the portion of the claim; routing the claim to the second payor when a confidence score value magnitude exceeds a first confidence threshold; and routing the claim to the first payor with a flag indicating the second payor may be responsible for the at least the portion of the claim when the confidence score value magnitude is between the first confidence threshold and a second confidence threshold. A magnitude of the confidence score value is greater with increased likelihood of the second payor being responsible for the at least a portion of the claim and a magnitude of the first confidence threshold is greater than a magnitude of the second confidence threshold.

In still other embodiments, assessing the likelihood that the first payor is not liable for the entirety of the claim and identifying the second payor comprises: generating a payor liability model based on adjudication of historical claims in which associations are determined between a plurality of patient coverage factors and responsible payors. The plurality of patient coverage factors comprises: an age of the patient, a spousal relationship status of the patient, information from historic claims associated with the patient, employment information for the patient and/or a spouse of the patient, an income level of the patient, and/or stored payor eligibility information for the patient.

In still other embodiments, generating the payor liability model comprises: using an Artificial Intelligence (AI) system to learn the associations between the plurality of patient coverage factors and the responsible payors.

In some embodiments of the inventive concept, a method comprises: receiving a patient insurance coverage eligibility request associated with a patient and destined for a first payor from a provider; assessing a likelihood that the patient is eligible for insurance coverage from a second payor; identifying the second payor responsive to the likelihood that patient is eligible for insurance coverage from a second payor exceeding a liability threshold; and notifying the provider that the patient is eligible for insurance coverage from the second payor.

In further embodiments, assessing the likelihood that the patient is eligible for insurance coverage from the second payor comprises: assessing the likelihood that the patient is eligible for insurance coverage from the second payor based on a plurality of patient coverage factors. The plurality of patient coverage factors comprises: an age of the patient, a spousal relationship status of the patient, information from historic claims associated with the patient, employment information for the patient and/or a spouse of the patient, an income level of the patient, and/or stored payor eligibility information for the patient.

In still further embodiments, identifying the second payor comprises: identifying the second payor based on a plurality of payor identification factors. The plurality of payor identification factors comprises: an age of the patient, a spousal relationship status of the patient, information from historic claims associated with the patient, employment information for the patient and/or a spouse of the patient, an income level of the patient, and/or stored payor eligibility information for the patient.

In still further embodiments, the method further comprises: confirming with the second payor that the patient is eligible for insurance coverage from the second payor.

In some embodiments of the inventive concept, a system comprises a processor; and a memory coupled to the processor and comprising computer readable program code embodied in the memory that is executable by the processor to perform operations comprising: receiving a claim associated with a patient and destined for a first payor from a provider; assessing a likelihood that the first payor is not liable for the entirety of the claim; identifying a second payor responsive to the likelihood that the first payor is not liable for the entirety of the claim exceeding a liability threshold; and routing the claim to the first payor, to the second payor, or to the provider based on routing rules provided by the first payor.

In other embodiments, assessing the likelihood that the first payor is not liable for the entirety of the claim comprises: assessing the likelihood that the first payor is not liable for the entirety of the claim based on a plurality of patient coverage factors. Identifying the second payor comprises: identifying the second payor based on a plurality of payor identification factors. The plurality of patient coverage factors and the plurality of payor identification factors comprise: an age of the patient, a spousal relationship status of the patient, information from historic claims associated with the patient, employment information for the patient and/or a spouse of the patient, an income level of the patient, and/or stored payor eligibility information for the patient.

In some embodiments of the inventive concept, a computer program product, comprises: a non-transitory computer readable storage medium comprising computer readable program code embodied in the medium that is executable by a processor to perform operations comprising: receiving a claim associated with a patient and destined for a first payor from a provider; assessing a likelihood that the first payor is not liable for the entirety of the claim; identifying a second payor responsive to the likelihood that the first payor is not liable for the entirety of the claim exceeding a liability threshold; and routing the claim to the first payor, to the second payor, or to the provider based on routing rules provided by the first payor.

It is noted that aspects described with respect to one embodiment may be incorporated in different embodiments although not specifically described relative thereto. That is, all embodiments and/or features of any embodiments can be combined in any way and/or combination. Moreover, other methods, systems, articles of manufacture, and/or computer program products according to embodiments of the inventive concept will be or become apparent to one with skill in the art upon review of the following drawings and detailed description. It is intended that all such additional systems, methods, articles of manufacture, and/or computer program products be included within this description, be within the scope of the present inventive subject matter and be protected by the accompanying claims.

### BRIEF DESCRIPTION OF THE DRAWINGS

Other features of embodiments will be more readily understood from the following detailed description of specific embodiments thereof when read in conjunction with the accompanying drawings, in which:

FIG. 1 is a block diagram that illustrates a communication network including a third party liability system for determining third party liability for a claim in accordance with some embodiments of the inventive concept;

FIG. 2 is a flowchart that illustrates operations for determining third party liability for a claim in accordance with some embodiments of the inventive concept;

FIG. 3 is a block diagrams that illustrates the third party liability system in accordance with some embodiments of the inventive concept;

FIG. 4 is a flowchart that illustrates operations for determining third party liability for a claim in accordance with further embodiments of the inventive concept;

FIG. 5 is a block diagrams that illustrates the third party liability system in accordance with further embodiments of the inventive concept;

FIGS. 6 and 7 are flowcharts that illustrate operations for determining third party liability for a claim in accordance with further embodiments of the inventive concept;

FIG. 8 is a data processing system that may be used to implement a third party liability system for determining third party liability for a claim in accordance with some embodiments of the inventive concept; and

FIG. 9 is a block diagram that illustrates a software/hardware architecture for use in in a third party liability system for determining third party liability for a claim in accordance with some embodiments of the inventive concept.

### DETAILED DESCRIPTION

In the following detailed description, numerous specific details are set forth to provide a thorough understanding of embodiments of the inventive concept. However, it will be understood by those skilled in the art that embodiments of the inventive concept may be practiced without these specific details. In some instances, well-known methods, procedures, components, and circuits have not been described in detail so as not to obscure the inventive concept. It is intended that all embodiments disclosed herein can be implemented separately or combined in any way and/or combination. Aspects described with respect to one embodiment may be incorporated in different embodiments although not specifically described relative thereto. That is, all embodiments and/or features of any embodiments can be combined in any way and/or combination.

As used herein, the term “provider” may mean any person or entity involved in providing health care products and/or services to a patient.

Embodiments of the inventive concept are described herein in the context of a third party liability system for determining third party liability for a claim that may include an artificial intelligence (AI) engine, which uses machine learning. It will be understood that embodiments of the inventive concept are not limited to a machine learning implementation of the third party liability system and other types of AI systems may be used including, but not limited to, a multi-layer neural network, a deep learning system, a natural language processing system, and/or computer vision system. Moreover, it will be understood that the multi-layer neural network is a multi-layer artificial neural network comprising artificial neurons or nodes and does not include a biological neural network comprising real biological neurons.

Some embodiments of the inventive concept stem from a realization that the process in which a payor pays a claim first and then determines, typically through a manual post-payment audit or fraud and abuse review, whether the payment was proper or whether one or more third parties, i.e., additional payors, may be responsible for at least a portion of the claim is often expensive and labor intensive. Some embodiments of the inventive concept may provide a third party liability system in which an intermediary located in the cloud, such as a clearinghouse for processing claims generated by providers, may be configured to receive and analyze claims before they reach the payors to determine, for each of the claims, whether the claim may have one or more additional payors that may be responsible for at least a portion of the claim. The third party liability system may assess a likelihood that a first payor (i.e., a payor to which the claim is addressed) is not liable for the entirety of the claim. One or more second payors may then be identified when the likelihood that the first payor is not liable for the entirety of the clam exceeds a liability threshold. The patient coverage factors and/or the patient identification factors that may be used in assessing the likelihood that the first payor is not liable for the entirety of the claim and/or the identity of one or more second payors that may be liable for at least a portion of the claim may include, but are not limited to, an age of the patient, a spousal relationship status of the patient, information from historic claims associated with the patient (e.g., CPT codes, provider names, patient demographic information, etc.), employment information for the patient and/or a spouse of the patient, an income level of the patient, and/or stored payor eligibility information for the patient. When one or more additional payors are identified that may be responsible as at least partial payors for a claim, their responsibility may be confirmed. For example, a patient insurance coverage eligibility request may be sent to a payor to confirm that the patient has coverage with that payor. When there are multiple potential additional payors that may be responsible for a claim, a confidence score value may be assigned to each of these payors that is based on the likelihood that the payor is responsible for a portion of the claim where the higher the magnitude of the confidence score value the greater the likelihood is of the payor being responsible for at least a portion of the claim. Patient insurance coverage eligibility requests may be communicated only to those payors having a confidence score value magnitude that exceeds a confidence threshold.

According to some embodiments of the inventive concept, the claim can be routed to the first payor, one or more second or additional payors, and/or to the provider based on routing rules provided by the first payor. For example, the claim may be routed to the first payor with a flag that informs that first payor that one or more second payors are responsible for at least a portion of the claim. In some embodiments, the flag may include a confidence level with respect to the responsibility of one or more other payors and may include the identities of these other payor(s). The claim may also be routed to the provider notifying the provider that the first payor is not responsible for at least a portion of the claim. In some embodiments, routing the claim to the provider may be in the form of a rejection placing the burden on the provider to route the claim to the proper payor. The claim may be routed to one or more second payors and a notification may be communicated to the first payor informing the first payor that the claim has been routed to one or more second payors. In some embodiments, the routing operation may be based on confidence score values assigned to the one or more second payors, respectively. For example, the confidence score value may be indicative of the likelihood that a second or additional payor is responsible for at least a portion of a claim where the higher the magnitude of the confidence score value the greater the likelihood is of a second or additional payor being responsible for at least a portion of the claim. A claim may be routed to a second or additional payor when the confidence score value magnitude exceeds a first confidence threshold, and the claim may be routed to the first payor with a flag indicating that a second or additional payor may be responsible for at least a portion of the claim when the confidence score value magnitude is between the first confidence threshold and a second confidence threshold magnitude less than the first confidence threshold magnitude.

In accordance with various embodiments of the inventive concept, the third party liability system may use a variety of techniques for assessing the likelihood that the first payor is not liable for the entirety of a claim and/or identifying one or more second payors including, for example, generating a payor liability model by using an Artificial Intelligence (AI) system to learn associations between patient coverage factors and responsible payors based on adjudication of historical claims. The patient coverage factors may include, but are not limited to, an age of the patient, a spousal relationship status of the patient, information from historic claims associated with the patient (e.g., CPT codes, provider names, patient demographic information, etc.), employment information for the patient and/or a spouse of the patient, an income level of the patient, and/or stored payor eligibility information for the patient.

Example embodiments may perform this claim analysis to reduce the number of claims that a payor discovers during a post payment audit or fraud and abuse review that may have another payor that is at least partially responsible for the charges contained thereon. Other example embodiments may address this issue at the time a provider requests a confirmation from a first payor that a patient has insurance coverage. The third party liability system may, in response to receiving an eligibility request transaction for a patient from a provider, assess a likelihood that the patient is eligible for insurance from one or more second payors, identify one or more second payors when the likelihood that the patient is eligible for insurance from the one or more second payors exceeds a liability threshold, and confirm with the one or more second payors that the patient is indeed eligible for insurance coverage, all in a similar manner as described above with respect to performing these operations in response to receiving a claim from a provider. In response to the patient insurance coverage eligibility request, the provider may be notified that the patient is eligible for insurance coverage from one or more second payors.

Thus, a third party liability system according to some embodiments of the inventive concept may improve efficiency and reduce costs for payors when manually reviewing claims as part of audits and/or fraud and abuse inquiries as the number of claims found for which another payor is at least partially responsible may be reduced. This may also reduce the costs associated with contacting the providers and/or other payors to request reimbursement for claim charges for which the payor is not responsible.

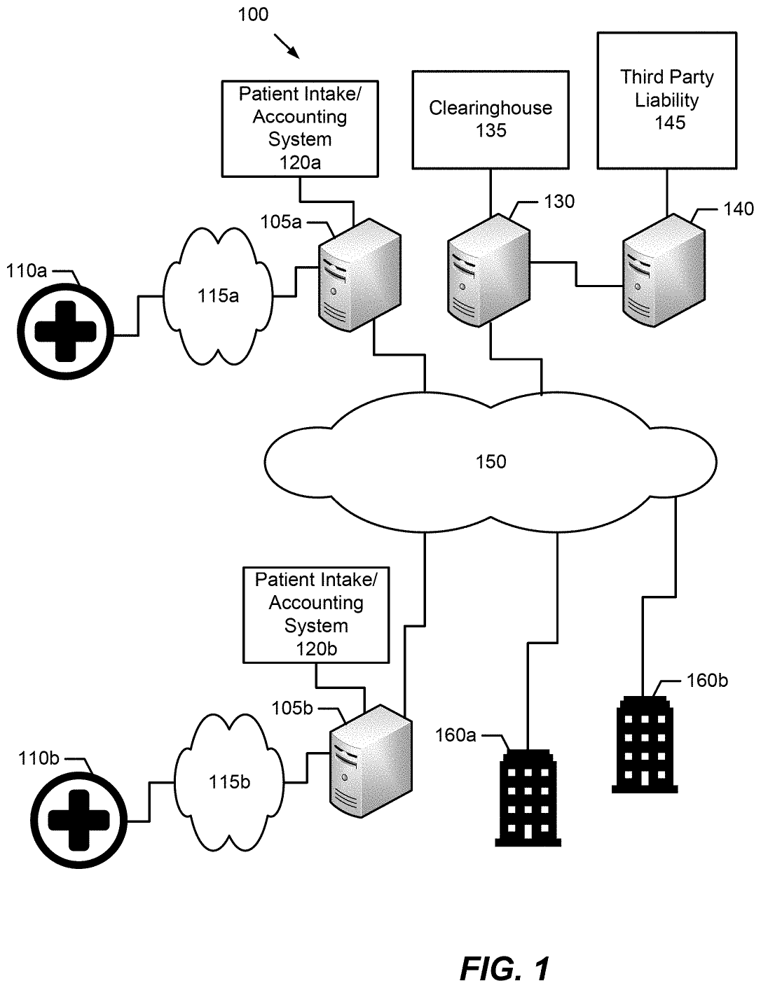

Referring to FIG. 1, a communication network 100 including a third party liability system for determining third party liability for a claim, in accordance with some embodiments of the inventive concept, comprises multiple health care provider facilities or practices 110a, 110b. Each health care provider facility or practice may represent various types of organizations that are used to deliver health care services to patients via health care professionals, which are referred to generally herein as “providers.” The providers may include, but are not limited to, hospitals, medical practices, mobile patient care facilities, diagnostic centers, lab centers, pharmacies, and the like. The providers may operate by providing health care services for patients and then invoicing one or more payors 160a and 160b for the services rendered. The payors 160a and 160b may include, but are not limited to, providers of private insurance plans, providers of government insurance plans (e.g., Medicare, Medicaid, state, or federal public employee insurance plans), providers of hybrid insurance plans (e.g., Affordable Care Act plans), providers of private medical cost sharing plans, and the patients themselves. Two provider facilities 110a, 110b are illustrated in FIG. 1 with the first provider including a first patient intake/accounting system server 105a accessible via a network 115a. The first patient intake/accounting system server 105a may be configured with a patient intake/accounting system module 120a to manage the intake of patients for appointments and to generate invoices for payors for services and products rendered through the provider 110a. The network 115a communicatively couples the first patient intake/accounting system server 105a to other devices, terminals, and systems in the provider's facility 110a. The network 115a may comprise one or more local or wireless networks to communicate with first patient intake/accounting system server 105a when the first patient intake/accounting system server 105a is located in or proximate to the health care service provider facility 110a. When the first patient intake/accounting system server 105a is in a remote location from the health care facility, such as part of a cloud computing system or at a central computing center, then the network 115a may include one or more wide area or global networks, such as the Internet. The second provider facility 110b is similar to the first provider facility 110a and includes a second patient intake/accounting system server 105b, which is configured with a patient intake/accounting system server 120b. The second patient intake/accounting system server 105b is coupled to other devices, terminals, and systems in the provider's facility 110b via a network 115b.

According to embodiments of the inventive concept, an intermediary may be used between a health care service provider and a payor for determining whether a third party may be at least partially liable for claims communicated to the payor. An intermediary server 130 may include a clearinghouse system module 135 that may be configured to receive incoming claims and/or patient insurance coverage eligibility requests from one or more providers 110a, 110b and route those claims and/or patient insurance coverage eligibility requests t one or more payors 160a, 160b. According to embodiments of the inventive concept, thee intermediary may further include a claim processing server/eligibility request server 140 that includes a third party liability module 145. The third party liability module 145 may be configured to intercept a claim received by the intermediary server 130, assess a likelihood that the payor 160a, 160b to whom the claim and/or insurance coverage eligibility request is destined is not liable for the entirety of the claim and/or that the patient may have coverage through other payors 160a, 160b, confirm with one or more of the other payors 160a, 160b whether they are responsible for at least a portion of the claim and/or whether they provide coverage to the patient, and route the claim to the payor 160a, 160b to whom the claim is directed, one or more other payors 160a, 160b, and/or the provider 110a, 110b based on rules provided by, for example, the payor 160a, 160b to whom the claim is directed. The intermediary server 130, the clearinghouse system module 135, the claim processing/eligibility request server 140, and the third party liability module 145 may be viewed collectively as a third party liability system for determining third party liability for a claim in accordance with some embodiments of the inventive concept.

A network 150 couples the patient intake/accounting system servers 105a, 105b to the intermediary server 130 and couples the payors 160a and 160b to the intermediary server 130. The network 150 may be a global network, such as the Internet or other publicly accessible network. Various elements of the network 150 may be interconnected by a wide area network, a local area network, an Intranet, and/or other private network, which may not be accessible by the general public. Thus, the communication network 150 may represent a combination of public and private networks or a virtual private network (VPN). The network 150 may be a wireless network, a wireline network, or may be a combination of both wireless and wireline networks.

The third party liability service provided through the intermediary server 130, the clearinghouse system module 135, the claim processing/eligibility request server 140, and the third party liability module 145 for determining third party liability for a claim may, in some embodiments, be embodied as a cloud service. For example, health care service providers and/or payors may access the third part liability system as a Web service. In some embodiments, the third party liability system service may be implemented as a Representational State Transfer Web Service (RESTful Web service).

Although FIG. 1 illustrates an example communication network including a third party liability system for determining third party liability for a claim, it will be understood that embodiments of the inventive subject matter are not limited to such configurations, but are intended to encompass any configuration capable of carrying out the operations described herein.

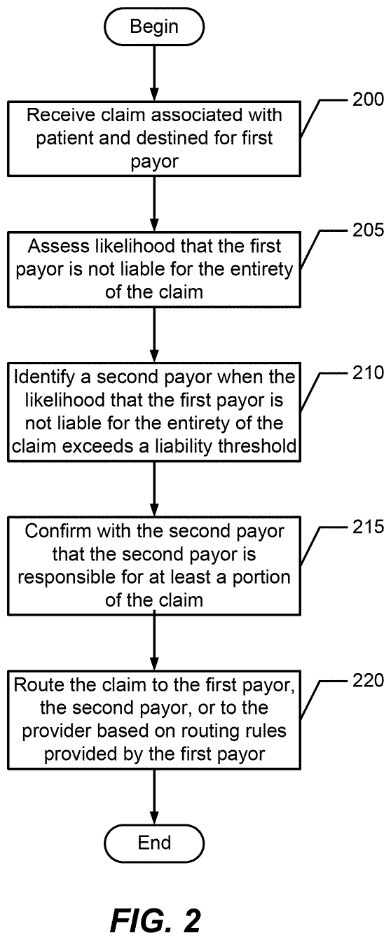

FIG. 2 is a flowchart that illustrates operations for determining third party liability for a claim in accordance with some embodiments of the inventive concept. Referring now to FIG. 2, operations begin at block 200 where a claim associated with a patient and destined for a first payor is received. As noted above, in one embodiment of the inventive concept, the claim is received by an intermediary server 130 responsible for routing (via a clearinghouse module 135) claims from providers to payors. Before routing, however, an assessment is made at block 205 of the likelihood that the first payor is not liable for the entirety of the claim. In accordance with various embodiments of the inventive concept, this assessment may be based on a variety of patient coverage factors including, but not limited to, an age of the patient, a spousal relationship status of the patient, information from historic claims associated with the patient (e.g., CPT codes, provider name, patient demographic information, etc.), employment information for the patient and/or a spouse of the patient, an income level of the patient, and/or stored payor eligibility information for the patient. For example, rules may be generated that conclude if a person is over a certain age, then the person is likely to have Medicare insurance. Similarly, if a person has an income below a certain level, then the person is likely to have Medicaid insurance. The rules may use employment information to conclude that the patient is likely to have private insurance through a particular employer. This may be due in part to information collected for other patients employed by the same employer, for example. If the patient has a spouse, then the rules may conclude that the patient is likely to have insurance coverage provided by the spouse's employer based on, for example, the spouse's claims and/or other claims for patients employed by the spouse's employer. One or more second payors may be identified at block 210 when the likelihood that the first payor is not liable for the entirety of the claim exceeds a liability threshold. One or more patient coverage factors, such as an age of the patient, a spousal relationship status of the patient, information from historic claims associated with the patient, employment information for the patient and/or a spouse of the patient, an income level of the patient, and/or stored payor eligibility information for the patient, may be used as payor identification factors to identify the one or more second payors that may be liable for the claim. In one embodiment, a blockchain ledger containing primary and/or secondary coverage information for a patient may be used as a factor or resource in assessing a likelihood that the first payor is not liable for the entirety of the claim and/or for identifying one or more second payors that may be responsible for at least a portion of the claim.

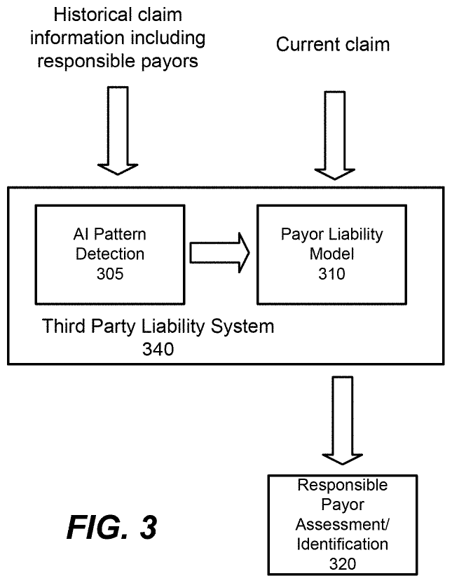

FIG. 3 is a block diagram that illustrates embodiments of the third party liability system in which an AI engine is used to assess whether one or more additional or alternative payors may be responsible for a claim and to identify such additional or alternative payors. As shown in FIG. 3, the third party liability system 340 includes an AI engine, which may be a machine learning engine comprising an AI pattern detection module 305 and a payor liability model 310. The AI pattern detection module 305 is configured to receive historical claim information, which may include, but is not limited to, CPT codes, provider names, patient names/identifications, patient demographic information, and responsible payor information, and may learn associations between the historical claim information and who the one or more responsible payors are for each claim. The AI pattern detection module 305 may then generate a payor liability model 310 based on these learned associations, which can be used to process a current claim, which may be accompanied by one or more of the above-described patient coverage factors and payor identification factors, such as an age of the patient, a spousal relationship status of the patient, information from categories that overlap with information contained in historic claims associated with the patient, employment information for the patient and/or a spouse of the patient, an income level of the patient, and/or stored payor eligibility information for the patient to determine potential third party liability for the claim, i.e., if one or more additional or second payors other than the first payor to which the claim is addressed, may be responsible for at least a portion of the claim. An assessment of the likelihood of one or more additional payors and/or identifications of these payors 320 may be output for use in confirming responsibility with these additional payors and/or routing the claim to one or more of these additional payors, the first payor to which the claim is addressed, and/or the provider.

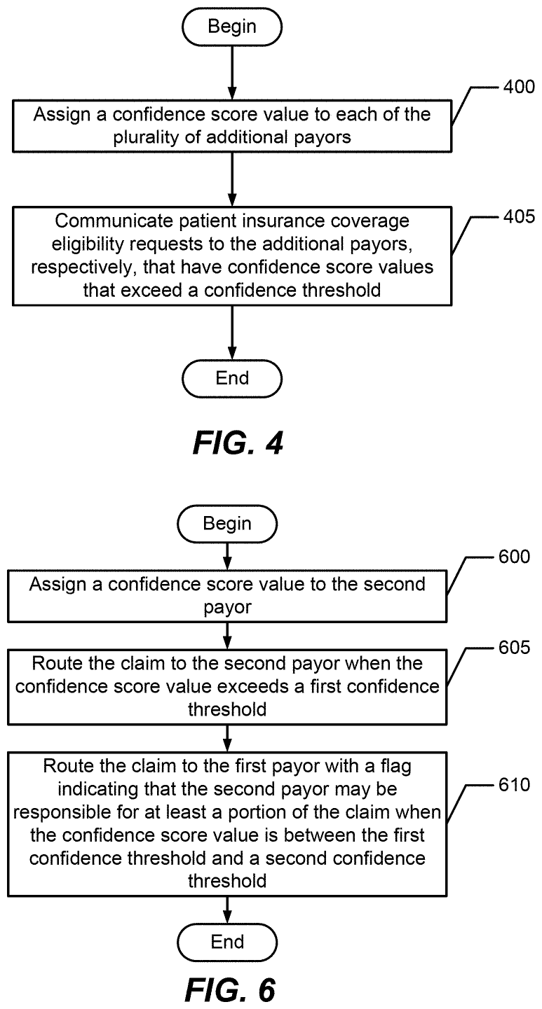

Returning to FIG. 2, when one or more second payors are identified as potentially being responsible for at least a portion of the claim, operations continue at block 215 where these second payor(s) may be contacted to confirm their responsibility. Other embodiments, however, of the third party liability system may not seek confirmation from one or more second payors before routing the claim to the first payor, one or more second payors, and/or the provider. The confirmation process may involve sending the potential payor a patient insurance coverage eligibility request. When multiple potential payors are identified, however, it may be more efficient to confirm eligibility only for those payors most likely to be liable. Referring now to FIG. 4, a confidence score may be assigned to each of a plurality of additional payors that may be liable for at least a portion of the claim. The magnitude of the confidence score is greater with increased likelihood that the payor is at least partially responsible. Patient insurance coverage eligibility requests may be communicated at block 405 only to those additional payors that have confidence score values of a magnitude that exceed a confidence threshold. An insurance coverage confirmation reply may be received from a payor in response to the patient insurance coverage eligibility request confirming that the patient has insurance coverage provided by the payor. A payor may also reply with a notification that the patient does not have insurance coverage with the payor.

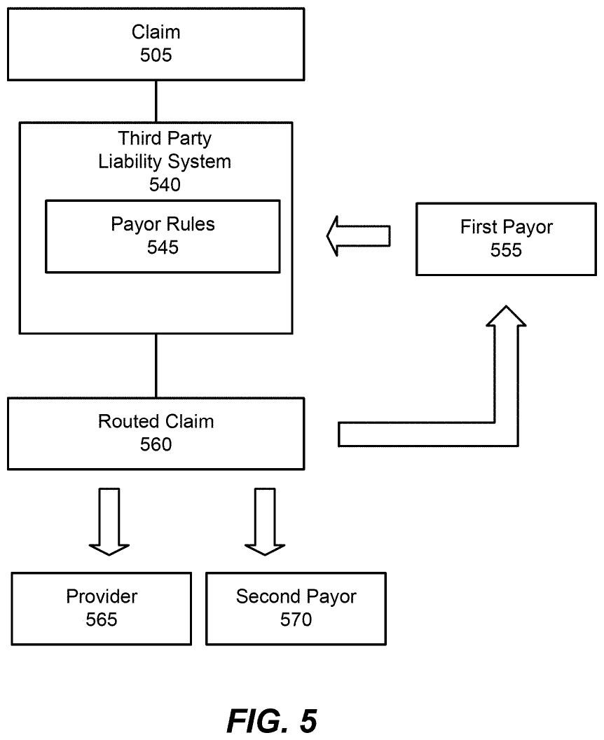

Operations continue at block 220 of FIG. 2 where the claim is routed to the first payor, one or more second payors, or to the provider based on routing rules provided by the first payor, i.e., the payor to whom the claim is addressed. FIG. 5 is a block diagram that illustrates embodiments of the third party liability system in which payor rules are used to determine how to route a claim after determining payor liability for the claim. Referring to FIG. 5, a claim 505 is provided to the third party liability system 540. The third party liability system 540 is configured with payor rules 545 that are provided by a first payor 555, i.e., a payor to whom the claim is addressed for payment. These payor rules 545 may specify the preferences of the first payor with respect to routing the claim based on the payor liability determined for the claim. The routed claim 560 may be communicated to the first payor 555, the provider 565, and/or one or more second or additional payors 570 in accordance with the preferences provided by the first payor 555 in the form of the payor rules 545.

Thus, returning to block 220 of FIG. 2, in accordance with the payor rules 545 provided by the first payor 555 of FIG. 5, the claim may be routed to various combinations of parties in accordance with different embodiments of the inventive concept. For example, the claim may be routed to the first payor with a flag that identifies the second payor as being responsible for at least a portion of the claim. The flag may include a confidence level with respect to the responsibility of the second payor and may include the identity of the second payor. In other embodiments, the claim may be provided to the provider notifying the provider that the first payor is not responsible for the claim. The notification to the provider may be in the form of a rejection of the claim thereby placing the burden on the provider to route the claim to the appropriate payor. In still other embodiments, the claim may be routed to one or more second payors for processing. In some embodiments, the first payor and/or the provider may receive notification that the claim is being routed to one or more second payors for processing. In still other embodiments, the claim may be routed to both the first payor and one or more second payors to allow each payor to process the claim and account for the respective payor's responsible portion of the claim. Notifications may again be provided to the provider, the first payor, and/or the one or more second payors notifying them of how the claim was routed and which parties are processing the claim.

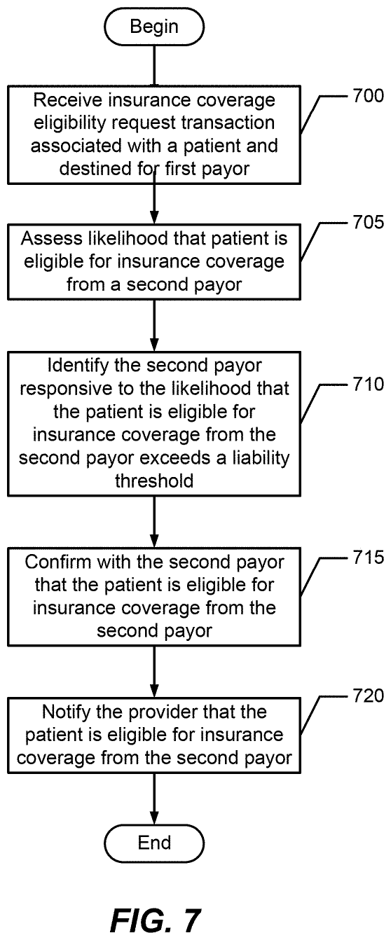

When one or more second or additional payors are identified as potentially liable for a claim, even though confirmation(s) are received that the patient has insurance coverage with these payor(s) there may still be a relatively low likelihood that each of these payor(s) is ultimately responsible for a portion of the claim. To improve efficiency in routing a claim, the claim may only be routed to those payors that are most likely to be responsible for the claim. In this regard, referring now to FIG. 6, operations begin at block 600 where a confidence score value is assigned to each of the secondary or additional payors based on a likelihood that the secondary or additional payor is responsible for at least a portion of the claim. The magnitude of the confidence score value is greater with increased likelihood that the payor is responsible for at least a portion of the claim. The claim may be routed to one or more second payors when their confidence score values exceed a first confidence threshold at block 605. The claim may be routed to the first payor with a flag indicated the second payor may be responsible for at least a portion of the claim when the confidence score value magnitude is between the first confidence threshold and a second confidence threshold at block 610 where the second confidence threshold magnitude is less than the first confidence threshold magnitude.

Example embodiments of the inventive concept have been described above with respect to analyzing a claim once it has already been prepared and communicated by a provider to determine whether one or more secondary or additional payors may be responsible for at least a portion thereof. Other example embodiments may determine if a patient may have one or more secondary or additional payors that are at least partially responsible responsible for charges associated with the patient's health care at the time a provider requests confirmation from a first payor that the patient has insurance coverage. Referring to FIG. 7, operations begin at block 700 where an insurance coverage eligibility request transaction is received that is associated with a patient and is destined for a first payor. An assessment is made at block 705 whether the patient is eligible for insurance coverage from one or more second payors. One or more second payors may be identified at block 710 when the likelihood that the patient is eligible for insurance coverage from the one or more second payors exceeds a liability threshold. The one or more second payors may be contacted using, for example, a patient insurance coverage eligibility request, to confirm that the one or more second payors provide insurance coverage to the patient at block 715. Example embodiment of such confirmation operations are described above with respect to FIG. 2, block 215 and FIG. 4. The provider may then be notified at block 720 that the patient is eligible for insurance coverage from one or more second payors responsive to the one or more second payors confirming that the patient has insurance coverage.

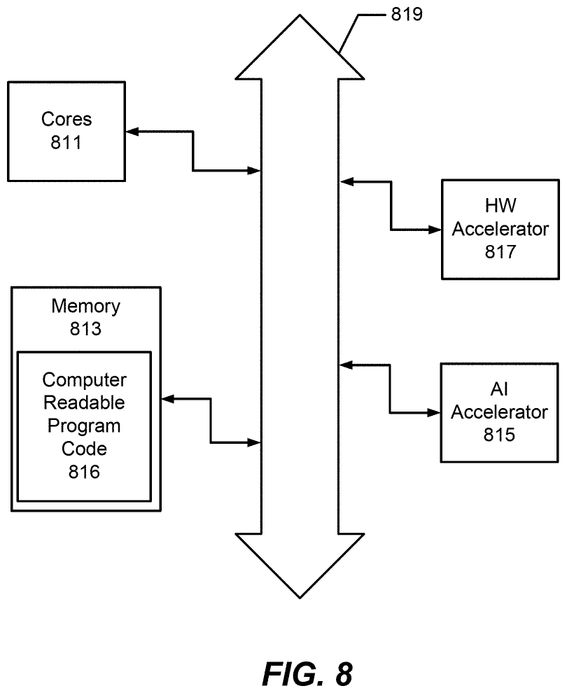

FIG. 8 is a block diagram of a data processing system that may be used to implement the claim processing/eligibility request server 140 of FIG. 1 and/or the third party liability systems 340 and 540 of FIGS. 3 and 5 in accordance with some embodiments of the inventive concept. As shown in FIG. 8, the data processing system may include at least one core 811, a memory 813, an artificial intelligence (AI) accelerator 815, and a hardware (HW) accelerator 817. The at least one core 811, the memory 813, the AI accelerator 815, and the HW accelerator 817 may communicate with each other through a bus 819.

The at least one core 811 may be configured to execute computer program instructions. For example, the at least one core 811 may execute an operating system and/or applications represented by the computer readable program code 816 stored in the memory 813. In some embodiments, the at least one core 811 may be configured to instruct the AI accelerator 815 and/or the HW accelerator 817 to perform operations by executing the instructions and obtain results of the operations from the AI accelerator 815 and/or the HW accelerator 817. In some embodiments, the at least one core 811 may be an ASIP customized for specific purposes and support a dedicated instruction set.

The memory 813 may have an arbitrary structure configured to store data. For example, the memory 813 may include a volatile memory device, such as dynamic random-access memory (DRAM) and static RAM (SRAM), or include a non-volatile memory device, such as flash memory and resistive RAM (RRAM). The at least one core 811, the AI accelerator 815, and the HW accelerator 817 may store data in the memory 813 or read data from the memory 813 through the bus 819.

The AI accelerator 815 may refer to hardware designed for AI applications. In some embodiments, the AI accelerator 815 may include a machine learning engine configured to determine third part liability for a claim. The AI accelerator 815 may generate output data by processing input data provided from the at least one core 815 and/or the HW accelerator 817 and provide the output data to the at least one core 811 and/or the HW accelerator 817. In some embodiments, the AI accelerator 815 may be programmable and be programmed by the at least one core 811 and/or the HW accelerator 817. The HW accelerator 817 may include hardware designed to perform specific operations at high speed. The HW accelerator 817 may be programmable and be programmed by the at least one core 811.

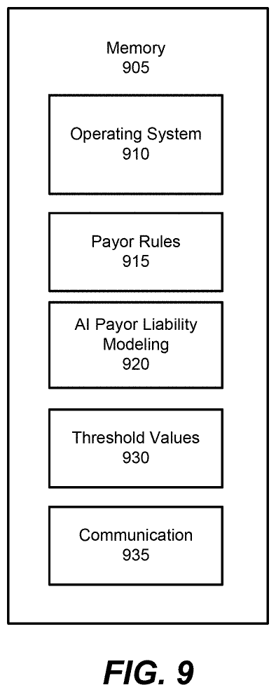

FIG. 9 illustrates a memory 905 that may be used in embodiments of data processing systems, such as the claim processing/eligibility request server 140 of FIG. 1, the third party liability systems 340 and 540 of FIGS. 3 and 5, and the data processing system of FIG. 8, respectively, to facilitate determining third party liability for a claim. The memory 905 is representative of the one or more memory devices containing the software and data used for facilitating operations of the claims processing/eligibility request server 140 and the third party liability module 145 as described herein. The memory 905 may include, but is not limited to, the following types of devices: cache, ROM, PROM, EPROM, EEPROM, flash, SRAM, and DRAM. As shown in FIG. 9, the memory 905 may contain five or more categories of software and/or data: an operating system 910, payor rules 915, an AI payor liability modeling module 920, threshold values 930, and a communication module 935. In particular, the operating system 910 may manage the data processing system's software and/or hardware resources and may coordinate execution of programs by the processor.

The payor rules module 915 may provide the payor rules 545 for determining how to route a claim based on payor preferences as described above with respect to FIG. 2, block 220 and FIG. 5. The AI payor liability modeling module 920 may be configured to perform one or more of the operations described above with respect to the third party liability system 340 of FIG. 3. The threshold values 930 may provide the thresholds, such as the liability and confidence thresholds for identifying one or more secondary or additional payors. The communication module 935 may be configured to facilitate communication between the claim processing/eligibility request server 140 of FIG. 1 and/or the third party liability systems 340 and 540 of FIGS. 3 and 5 and the providers 110a, 110b and payors 160a, 160b of FIG. 1.

Although FIGS. 8 and 9 illustrate hardware/software architectures that may be used in data processing systems, such as the claim processing/eligibility request server 140 of FIG. 1, the third party liability systems 340 and 540 of FIGS. 3 and 5, and the data processing system of FIG. 8, respectively, in accordance with some embodiments of the inventive concept, it will be understood that the present invention is not limited to such a configuration but is intended to encompass any configuration capable of carrying out operations described herein.

Computer program code for carrying out operations of data processing systems discussed above with respect to FIGS. 1-8 may be written in a high-level programming language, such as Python, Java, C, and/or C++, for development convenience. In addition, computer program code for carrying out operations of the present invention may also be written in other programming languages, such as, but not limited to, interpreted languages. Some modules or routines may be written in assembly language or even micro-code to enhance performance and/or memory usage. It will be further appreciated that the functionality of any or all of the program modules may also be implemented using discrete hardware components, one or more application specific integrated circuits (ASICs), or a programmed digital signal processor or microcontroller.

Moreover, the functionality of the intermediary server 130 of FIG. 1, the claim processing/eligibility request server 140 of FIG. 1, the third party liability systems 340 and 540 of FIGS. 3 and 5, and the data processing system of FIG. 8 may each be implemented as a single processor system, a multi-processor system, a multi-core processor system, or even a network of stand-alone computer systems, in accordance with various embodiments of the inventive concept. Each of these processor/computer systems may be referred to as a “processor” or “data processing system.” The functionality provided by the intermediary server 130 and the claim processing/eligibility server 140 may be merged into a single server or maintained as separate servers in accordance with different embodiments of the inventive concept.

The data processing apparatus described herein with respect to FIGS. 1-8 may be used to facilitate supplementing a claim with additional clinical information according to some embodiments of the inventive concept described herein. These apparatus may be embodied as one or more enterprise, application, personal, pervasive and/or embedded computer systems and/or apparatus that are operable to receive, transmit, process and store data using any suitable combination of software, firmware and/or hardware and that may be standalone or interconnected by any public and/or private, real and/or virtual, wired and/or wireless network including all or a portion of the global communication network known as the Internet, and may include various types of tangible, non-transitory computer readable media. In particular, the memory 905 when coupled to a processor includes computer readable program code that, when executed by the processor, causes the processor to perform operations including one or more of the operations described herein with respect to FIGS. 1-7.

Some embodiments of the inventive concept may provide a third party liability system may improve a payor's claim processing operations by reducing the payments made by a payor for which the payor is not liable. Payors traditionally pay a claim first and then discover through post payment audits or fraud and abuse reviews of paid claims that another payor is responsible in whole or in part of a paid claim. This review along with the process of contacting the provider and/or the other payor for reimbursement can be labor intensive and costly. The third party liability system according to embodiments of the inventive concept may screen claims before they reach the payor to identify those claims for which one or more other payors may be responsible for at least a portion of the charges contained therein. As a result, the claim can be forwarded to the payor with a notification that the payor is likely not responsible for the entirety of the claim, forward the claim to one or more of the other payors that are likely responsible for at least a portion of the claim, and/or notify the provider that the payor is likely not responsible for the entirety of the claim. This may reduce the number of claims for a payor that are discovered in post payment audits or fraud and abuse reviews to have been paid improperly due to one or more other payors being at least partially responsible. As a result, the costs associated with reviewing the claims to discover the payment errors and notifying the provider and/or the additional payors of the liability of the additional payors to obtain reimbursement may be reduced.

### Further Definitions and Embodiments

In the above-description of various embodiments of the present inventive concept, it is to be understood that the terminology used herein is for the purpose of describing particular embodiments only and is not intended to be limiting of the invention. Unless otherwise defined, all terms (including technical and scientific terms) used herein have the same meaning as commonly understood by one of ordinary skill in the art to which this inventive concept belongs. It will be further understood that terms, such as those defined in commonly used dictionaries, should be interpreted as having a meaning that is consistent with their meaning in the context of this specification and the relevant art and will not be interpreted in an idealized or overly formal sense expressly so defined herein.

The flowchart and block diagrams in the figures illustrate the architecture, functionality, and operation of possible implementations of systems, methods, and computer program products according to various aspects of the present inventive concept. In this regard, each block in the flowchart or block diagrams may represent a module, segment, or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

The terminology used herein is for the purpose of describing particular aspects only and is not intended to be limiting of the inventive concept. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Like reference numbers signify like elements throughout the description of the figures.

In the above-description of various embodiments of the present inventive concept, aspects of the present inventive concept may be illustrated and described herein in any of a number of patentable classes or contexts including any new and useful process, machine, manufacture, or composition of matter, or any new and useful improvement thereof. Accordingly, aspects of the present inventive concept may be implemented entirely hardware, entirely software (including firmware, resident software, micro-code, etc.) or combining software and hardware implementation that may all generally be referred to herein as a “circuit,” “module,” “component,” or “system.” Furthermore, aspects of the present inventive concept may take the form of a computer program product comprising one or more computer readable media having computer readable program code embodied thereon.

Any combination of one or more computer readable media may be used. The computer readable media may be a computer readable signal medium or a computer readable storage medium. A computer readable storage medium may be, for example, but not limited to, an electronic, magnetic, optical, electromagnetic, or semiconductor system, apparatus, or device, or any suitable combination of the foregoing. More specific examples (a non-exhaustive list) of the computer readable storage medium would include the following: a portable computer diskette, a hard disk, a random access memory (RAM), a read-only memory (ROM), an erasable programmable read-only memory (EPROM or Flash memory), an appropriate optical fiber with a repeater, a portable compact disc read-only memory (CD-ROM), an optical storage device, a magnetic storage device, or any suitable combination of the foregoing. In the context of this document, a computer readable storage medium may be any tangible medium that can contain or store a program for use by or in connection with an instruction execution system, apparatus, or device.

The description of the present inventive concept has been presented for purposes of illustration and description, but is not intended to be exhaustive or limited to the inventive concept in the form disclosed. Many modifications and variations will be apparent to those of ordinary skill in the art without departing from the scope and spirit of the inventive concept. The aspects of the inventive concept herein were chosen and described to best explain the principles of the inventive concept and the practical application, and to enable others of ordinary skill in the art to understand the inventive concept with various modifications as are suited to the particular use contemplated.

What is claimed is:

1. A method, comprising:
receiving a claim associated with a patient and destined for a first payor from a provider;
assessing a likelihood that the first payor is not liable for an entirety of the claim;
identifying a second payor responsive to the likelihood that the first payor is not liable for the entirety of the claim exceeding a liability threshold; and
routing the claim to the first payor, to the second payor, or to the provider based on routing rules provided by the first payor.

2. The method of claim 1, wherein assessing the likelihood that the first payor is not liable for the entirety of the claim comprises:
assessing the likelihood that the first payor is not liable for the entirety of the claim based on a plurality of patient coverage factors;
wherein the plurality of patient coverage factors comprises: an age of the patient, a spousal relationship status of the patient, information from historic claims associated with the patient, employment information for the patient and/or a spouse of the patient, an income level of the patient, and/or stored payor eligibility information for the patient.

3. The method of claim 1, wherein identifying the second payor comprises:
identifying the second payor based on a plurality of payor identification factors;
wherein the plurality of payor identification factors comprises: an age of the patient, a spousal relationship status of the patient, information from historic claims associated with the patient, employment information for the patient and/or a spouse of the patient, an income level of the patient, and/or stored payor eligibility information for the patient.

4. The method of claim 1, wherein identifying the second payor comprises:
identifying a plurality of additional payors based on a plurality of payor identification factors responsive to the likelihood that the first payor is not liable for the entirety of the claim exceeding a liability threshold;
wherein the plurality of payor identification factors comprises: an age of the patient, a spousal relationship status of the patient, information from historic claims associated with the patient, employment information for the patient and/or a spouse of the patient, an income level of the patient, and/or stored payor eligibility information for the patient.

5. The method of claim 4, further comprising:
confirming with the plurality of additional payors that the additional payors are responsible for at least portions of the claim, respectively.

6. The method of claim 5, wherein confirming with the plurality of additional payors that the additional payors are responsible for at least portions of the claim, respectively, comprises:
assigning a confidence score value to each of the plurality of additional payors based on a likelihood that the respective one of the additional payors is responsible for a respective portion of the claim; and
communicating patient insurance coverage eligibility requests to ones of the additional payors, respectively, having confidence score value magnitudes that exceed a confidence threshold;
wherein the magnitude of the confidence score value is greater with increased likelihood of being responsible for the respective portion of the claim.

7. The method of claim 1, further comprising:
confirming with the second payor that the second payor is responsible for at least a portion of the claim.

8. The method of claim 7, wherein confirming with the second payor that the second payor is responsible for at least the portion of the claim comprises:
communicating a patient insurance coverage eligibility request to the second payor; and
receiving an insurance coverage confirmation reply from the second payor for the patient when the patient has insurance coverage provided by the second payor.

9. The method of claim 1, wherein routing the claim to the first payor, to the second payor, or to the provider based on the routing rules provided by the first payor comprises:
routing the claim to the first payor with a flag that identifies the second payor as being responsible for at least the portion of the claim.

10. The method of claim 1, wherein routing the claim to the first payor, to the second payor, or to the provider based on the routing rules provided by the first payor comprises:
routing the claim to the provider notifying the provider that the first payor is not responsible for the entirety of the claim.

11. The method of claim 1, wherein routing the claim to the first payor, to the second payor, or to the provider based on the routing rules provided by the first payor comprises:
routing the claim to the second payor.

12. The method of claim 11, further comprising:
notifying the first payor that the claim has been routed to the second payor.

13. The method of claim 1, wherein routing the claim to the first payor, to the second payor, or to the provider based on the routing rules provided by the first payor comprises:
assigning a confidence score value to the second payor based on a likelihood that the second payor is responsible for the at least the portion of the claim;
routing the claim to the second payor when a confidence score value magnitude exceeds a first confidence threshold; and
routing the claim to the first payor with a flag indicating the second payor may be responsible for the at least the portion of the claim when the confidence score value magnitude is between the first confidence threshold and a second confidence threshold;
wherein the magnitude of the confidence score value is greater with increased likelihood of the second payor being responsible for the at least a portion of the claim; and
wherein a magnitude of the first confidence threshold is greater than a magnitude of the second confidence threshold.

14. The method of claim 1, wherein assessing the likelihood that the first payor is not liable for the entirety of the claim and identifying the second payor comprises:
generating a payor liability model based on adjudication of historical claims in which associations are determined between a plurality of patient coverage factors and responsible payors;
wherein the plurality of patient coverage factors comprises: an age of the patient, a spousal relationship status of the patient, information from historic claims associated with the patient, employment information for the patient and/or a spouse of the patient, an income level of the patient, and/or stored payor eligibility information for the patient.

15. The method of claim 14, wherein generating the payor liability model comprises:
using an Artificial Intelligence (AI) system to learn the associations between the plurality of patient coverage factors and the responsible payors.

16. A method, comprising:
receiving a patient insurance coverage eligibility request associated with a patient and destined for a first payor from a provider;
assessing a likelihood that the patient is eligible for insurance coverage from a second payor;
identifying the second payor responsive to the likelihood that patient is eligible for insurance coverage from a second payor exceeding a liability threshold; and
notifying the provider that the patient is eligible for insurance coverage from the second payor.

17. The method of claim 16, wherein assessing the likelihood that the patient is eligible for insurance coverage from the second payor comprises:
assessing the likelihood that the patient is eligible for insurance coverage from the second payor based on a plurality of patient coverage factors;
wherein the plurality of patient coverage factors comprises: an age of the patient, a spousal relationship status of the patient, information from historic claims associated with the patient, employment information for the patient and/or a spouse of the patient, an income level of the patient, and/or stored payor eligibility information for the patient.

18. The method of claim 16, wherein identifying the second payor comprises:
identifying the second payor based on a plurality of payor identification factors;
wherein the plurality of payor identification factors comprises: an age of the patient, a spousal relationship status of the patient, information from historic claims associated with the patient, employment information for the patient and/or a spouse of the patient, an income level of the patient, and/or stored payor eligibility information for the patient.

19. The method of claim 16, further comprising:
confirming with the second payor that the patient is eligible for insurance coverage from the second payor.

20. A system, comprising:
a processor; and
a memory coupled to the processor and comprising computer readable program code embodied in the memory that is executable by the processor to perform operations comprising:
receiving a claim associated with a patient and destined for a first payor from a provider;
assessing a likelihood that the first payor is not liable for an entirety of the claim;
identifying a second payor responsive to the likelihood that the first payor is not liable for the entirety of the claim exceeding a liability threshold; and
routing the claim to the first payor, to the second payor, or to the provider based on routing rules provided by the first payor.

21. The system of claim 20, wherein assessing the likelihood that the first payor is not liable for the entirety of the claim comprises:
assessing the likelihood that the first payor is not liable for the entirety of the claim based on a plurality of patient coverage factors;
wherein identifying the second payor comprises:
identifying the second payor based on a plurality of payor identification factors:
wherein the plurality of patient coverage factors and the plurality of payor identification factors comprise: an age of the patient, a spousal relationship status of the patient, information from historic claims associated with the patient, employment information for the patient and/or a spouse of the patient, an income level of the patient, and/or stored payor eligibility information for the patient.

22. A computer program product, comprising:
a non-transitory computer readable storage medium comprising computer readable program code embodied in the medium that is executable by a processor to perform operations comprising:
receiving a claim associated with a patient and destined for a first payor from a provider;
assessing a likelihood that the first payor is not liable for an entirety of the claim;
identifying a second payor responsive to the likelihood that the first payor is not liable for the entirety of the claim exceeding a liability threshold; and
routing the claim to the first payor, to the second payor, or to the provider based on routing rules provided by the first payor.
